(19) United States
(12) Patent Application Publication
(10) Pub. No.: US 2002/0188286 A1
(43) Pub. Date: Dec. 12, 2002

(54) Methods for treating vulnerable plaque

(76) Inventors: Rodolfo C. Quijano, Laguna Hills, CA (US); Hosheng Tu, Tustin, CA (US)

Correspondence Address: Hosheng Tu, 2151 Palermo, Tustin, CA, 92782, US

(21) Appl. No.: 09/876,283

(22) Filed: Jun. 6, 2001

(51) Int. Cl. A61B 18/18

(52) U.S. Cl. 606/20; 606/41; 606/34

(57) ABSTRACT

A method and devices for rendering a focal tissue of a vulnerable plaque less vulnerable in a patient, comprising cooling said vulnerable plaque. The cooling may be accomplished with a cryogenic catheter, wherein said cryogenic catheter is provided with a cryogenic fluid path to cool a distal contact section of the catheter. Alternately, the cryogenic catheter may comprise elements of different electromotive potential conductively connected at a tip junction, the method further comprising passing an electrical current through said elements to reduce a temperature of the tip junction in accordance with the Peltier effect adapted for thermoelectric cooling said vulnerable plaque.

### FIELD OF THE INVENTION

The present invention relates generally to medical device for treating tissue and methods of use, and more particularly, to medical devices and methods comprising cryo-therapy effective to render a vulnerable plaque less vulnerable toward rupture or erosion.

### BACKGROUND OF THE INVENTION

This invention relates to the treatment of vulnerable plaque in the cardiovascular circulation system. Vulnerable plaque is a subset of the atherosclerotic plaque and is generally characterized by its vulnerability for causing pathologic effects to the circulation system, such as thrombosis, embolism, clotting, occlusion, or vaso-constriction. The vulnerability of a vulnerable plaque may relate to rupture, erosion or other unknown reasons.

Typically, several plaques are found in arteries afflicted with this disease. In most cases of terminal coronary artery disease, only one or a few plaques rupture and lead to catastrophe. The challenge to detect a vulnerable plaque early is that the vulnerable plaque is often angiographically invisible and is asymptomatic. Several research programs are underway to develop means for detecting a vulnerable plaque, such as thermography, ultrasonic elastogram, intravascular ultrasound, near infrared spectrum, optic coherent tomography, magnetic resonance imaging and the like. Vulnerable plaques generally consist of a lipid-rich core (“lipid pool”) and inflammatory cells. The cap over the lipid pool is very thin, in the sub-millimeter range. The vulnerable plaques are at a significant risk of precipitating infarction clinically.

Casscells, III et al. in U.S. Pat. No. 5,906,636 discloses the pathophysiology of a vulnerable plaque and a method for treating inflamed atherosclerotic plaques by heating the cells for a sufficient time and at a sufficient temperature to induce apoptosis. The entire contents of this patent are incorporated herein by reference. Casscells, III et al. does not disclose a cryo-therapy for reducing or eliminating the vulnerability of a vulnerable plaque. Typically, a method for rendering a vulnerable plaque of a patient less vulnerable may comprise cooling the vulnerable plaque. It is based on a hypothesis that the cryo-therapy reduces the reactivity of the inflamed cells, and conceivably cools/solidifies the lipid core under the thin plaque cap so that the cryo-therapy might lead to mitigate, inhibit, or reverse the lipid-forming process of a vulnerable plaque.

Apoptosis is a form of programmed cell death in which the dying cells retain membrane integrity and are rapidly phagocytosed and digested by macrophages or by neighboring cells. The dead cells are rapidly cleared without leaking their contents and therefore little inflammatory reaction follows. It can be induced by the withdrawal of growth factors and to some extent by factors which can also cause necrosis such as extreme lack of oxygen or glucose, heat, oxidation and other physical factors.

Casscells, III et al. teaches a heat-therapy at a temperature range of about 38.5° C. to 44° C. for a treating period of about 15 to 60 minutes that induces programmed cell death. As is well known in the prostate or tumors treatment programs, cryo-therapy is equally effective as compared to the heat-therapy in treating tissue. The cryo-therapy has the advantage of causing little damage to the surrounding tissue even when the surrounding tissue is slightly cooled.

Campbell in U.S. Pat. No. 5,924,997 discloses a catheter and method for thermally mapping hot spots in vascular lesions. The entire contents of Campbell patent are incorporated herein by reference. Though Campbell discloses a thermal mapping technique with optionally ultrasonic imaging arrangement suitable for imaging the body vessel, he does not disclose a cryo-therapy for reducing the vulnerability of a vulnerable plaque.

Uretsky et al. (Circulation 2000;102:611-616) studies sudden unexpected death at autopsy in heart failure patients. They report that acute coronary findings are frequent and usually not clinically diagnosed in heart failure patients with coronary artery disease, particularly in those dying suddenly, suggesting the importance of acute coronary events as a trigger for sudden death in this setting. There is a clinical need to detect and treat a vulnerable plaque in a patient so as to reduce the vulnerability of the vulnerable plaques and the sudden death therefrom.

Goldstein et al. reports that almost 40% of patients who have an acute myocardial infarction have areas of multiple complex coronary plaque and these patients are likely to experience recurrent cardiac events (N Eng J Med 2000;343:915-922). The most common method is by administering drug systemically. The literature indicates that there seems no therapeutic method to locally treat multiple complex coronary plaque in situ. By applying the cryo-therapy of the present disclosure, a region of multiple complex coronary plaque may be treatable in situ.

Johnson et al. in U.S. Pat. No. 4,860,744 discloses a thermoelectrically controlled heat medical catheter. More particularly, Johnson et al discloses a system and methods for providing controlled heating or cooling of a small region of body tissue to effectuate the removal of tumors and deposits, such as atheromatous plaque. Though Johnson et al. teaches a medical catheter in accordance with the Peltier effect adapted for thermoelectric heating/cooling for destruction of diseased tissue and/or tumors in various parts of the body, Johnson et al. does not disclose a method for cooling and solidifying the lipid pool as means for rendering the vulnerable plaque less vulnerable to rupture.

Larsen et al. in U.S. Pat. No. 5,529,067 discloses methods and apparatus for use in procedures related to the electrophysiology of the heart, such as identifying or evaluating the electrical activity of the heart, diagnosing and/or treating conditions associated with the electrophysiology of the heart, entire contents of which are incorporated herein by reference. Specifically, they teach an apparatus having thermocouple elements of different electromotive potential conductively connected at a junction and reducing the temperature of the junction in accordance with the Peltier effect for cooling the contacted heart tissue. However, Larsen et al. does not disclose a method for rendering a vulnerable plaque of a patient less vulnerable comprising cooling said vulnerable plaque.

Rowland in U.S. Pat. No. 6,096,032 discloses a medical cryosurgical device comprising a primary heat extraction means for extracting heat from a coolant so as to achieve a low temperature of about −50 to −60° C. that is required for effective use as a cryo-surgical device. However, Roland does not teach a method and an apparatus for rendering a vulnerable plaque of a patient less vulnerable comprising cooling said vulnerable plaque.

Lehmann et al. in U.S. Pat. No. 6,235,019 discloses a cryosurgical catheter comprising a plurality of inner members that allow at least one cryogenic fluid path and at least one cooling point along its catheter body. However, Lehmann et al. does not teach a medical catheter and cryo-therapy method for rendering a vulnerable plaque of a patient less vulnerable comprising cooling said vulnerable plaque.

Therefore, there is an urgent clinical need for a medical device comprising incorporating therapeutic means at some focal target tissue to provide site-specific treatment. More particularly, there is a need for a medical catheter and methods for rendering a vulnerable plaque of a patient less vulnerable comprising cooling the vulnerable plaque.

### SUMMARY OF THE INVENTION

In general, it is an object of the present invention to provide a device and a method for rendering a vulnerable plaque of a patient less vulnerable comprising cooling the target vulnerable plaque. The temperature for cooling the vulnerable plaque is no higher than 36° C. More preferably, a temperature for cooling the vulnerable plaque is in the range of about 36° C. to −30° C. that is sufficient to cool and solidify the lipid pool of a vulnerable plaque.

It is another object of the present invention to provide a method for detecting said vulnerable plaque prior to the cryo-therapy cooling treatment. The technique for detecting said vulnerable plaque may include monitoring temperature of the vulnerable plaque.

In one embodiment, the method of cooling may be accomplished with a cryogenic catheter, wherein the cryogenic catheter is provided with a cryogenic fluid path to cool a distal contact section of the catheter, said distal contact section being configured to be positioned for contacting the vulnerable plaque intimately. The distal contact section is preferably configured as a concave configuration adapted for encircling the vulnerable plaque for a purpose of intimate contact. Other configurations are equally applicable depending on the shape of the vulnerable plaques.

In another embodiment, the method of cooling a tissue may be accomplished with a cryogenic catheter, said cryogenic catheter comprising elements of different electromotive potential conductively connected at a tip junction. The method may further comprise passing an electrical current through the elements to reduce a temperature of the tip junction in accordance with the Peltier effect adapted for thermoelectric cooling the vulnerable plaque. The tip junction is configured to be positioned for contacting the vulnerable plaque intimately.

It is still another object of the present invention to provide a medical catheter for rendering a vulnerable plaque of a patient less vulnerable. The medical catheter may comprise elements of different electromotive potential conductively connected at a thermoelectric tip junction disposed at a distal section of the medical catheter, wherein the thermoelectric tip junction is adapted for contacting said vulnerable plaque. There is a first electrical wire and a second electrical wire supplying an electrical current through the elements to reduce temperature of the tip junction in accordance with the Peltier effect adapted for thermoelectric cooling the vulnerable plaque.

The distal contact section or the tip junction of the present invention is suitably configured and adapted for encircling the vulnerable plaque for a purpose of intimate contact, wherein the configuration of the distal contact section or the tip junction may be selected from a group consisting of concave, convex, semicylindrical, circular, elliptic hyperboloid, ellipsoid, oblate spheroid, hyperbolic paraboloid, elliptic paraboloid, and irregular configuration.

### BRIEF DESCRIPTION OF THE DRAWINGS

Additional objects and features of the present invention will become more apparent and the invention itself will be best understood from the following Detailed Description of Exemplary Embodiments, when read with reference to the accompanying drawings.

FIG. 1 is an overall view of a medical catheter constructed in accordance with the principles of the present invention.

FIG. 2 is a cross-sectional view of the distal section of the medical catheter, showing elements of different electromotive potential conductively connected at a tip junction.

FIG. 3 is a longitudinal cross-sectional view of the distal end portion of an apparatus utilizing the Peltier effect.

FIG. 4 is a transverse cross-sectional view of a vulnerable plaque for illustration purposes.

FIG. 5 is a longitudinal cross-sectional view of a vulnerable plaque in a patient.

FIG. 6 is a perspective view of a blood vessel having a vulnerable plaque treated by a medical catheter of the present invention.

### DETAILED DESCRIPTION OF EXEMPLARY EMBODIMENTS

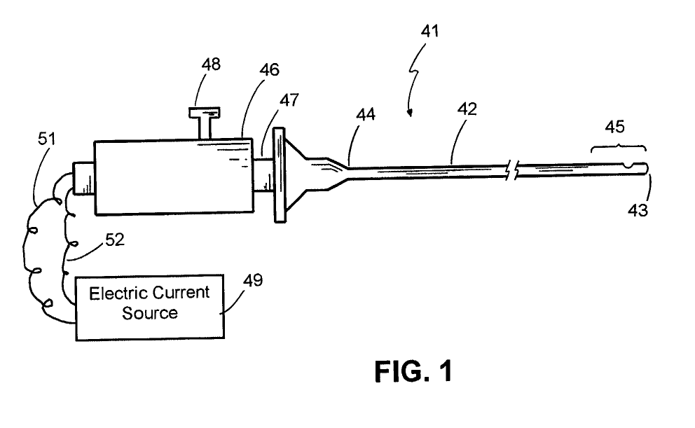

Referring to FIGS. 1 to 6, what is shown is an embodiment of a medical device having site-specific therapeutic ability and capability comprising cooling a vulnerable plaque effective for rendering the vulnerable plaque of a patient less vulnerable.

FIG. 1 shows an overall view of a medical catheter constructed in accordance with the principles of the present invention. The medical catheter comprises providing cooling to a contact portion at the distal section of the catheter and site-specifically delivering cryo-therapy to treat the vulnerable plaque. In one embodiment, the medical catheter 41 comprises a catheter body 42 having a distal end 43, a proximal end 44 and a distal section 45. A handle 46 is attached to the proximal end 44 of the catheter body. The catheter may be provided with an external electric current source 49 and two insulated conducting wires 51 and 52 for providing electrical current to a therapy element (heated or cooled) at the catheter distal section 45.

The catheter may also comprise a steerable mechanism 47 for steering the distal section 45 into a body conduit and also steering the therapeutic element contact point at the distal section firmly against the vulnerable plaque for treatment. A power switch control 48 may be provided to control the electrical current, the current flow direction, and other operating conditions.

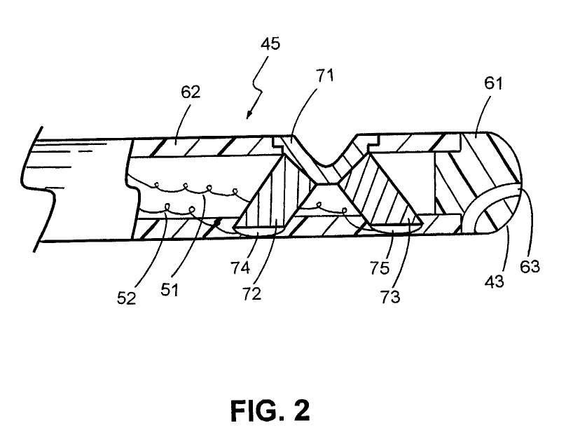

FIG. 2 shows a cross-sectional view of the distal section 45 of the medical catheter, showing elements 72, 73 of different electromotive potential conductively connected at a tip junction 71. The tip junction is adapted to serve as a therapy element of the present invention. An optional cap 61 is mounted at the distal end 43 of the catheter. Optionally, there is a guidewire lumen 43 located at the distal section 45 of the medical catheter 41 for the purposes of riding the catheter over a pre-deployed guidewire to the lesion location.

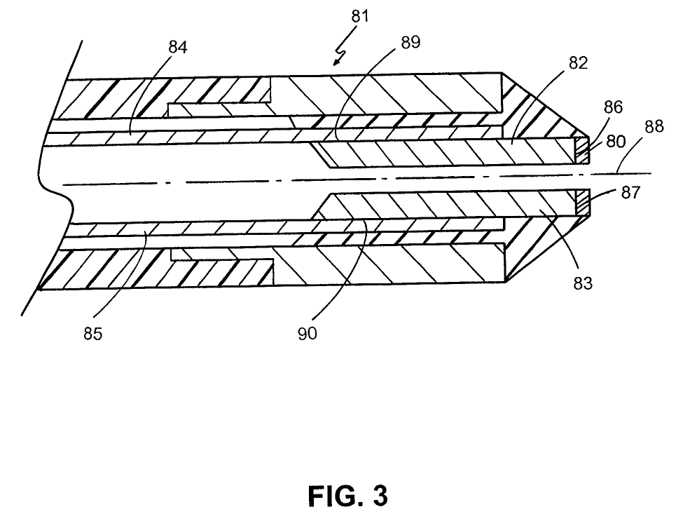

As described in U.S. Pat. Nos. 4,860,744, 5,529,067 and generally shown in FIG. 3 of the present invention, the therapeutic element 81 utilizes one pair of P (positive) and N (negative) thermoelectric elements or legs. The materials of the thermoelectric therapeutic element 81 may be configured in a variety of different ways such as bar or wire forms design. The P leg 82 and N leg 83 are electrically separated along their lengths, but are conductively joined at one end. The contact junctions 86, 87 represent one of junctions. With reference to U.S. Pat. No. 4,860,744, a molybdenum silicide plate to join the two legs at the contact junctions 86, 87 may be optionally used. The molybdenum silicide plate that connects the legs at the contact junction 86, 87 is referred to as a cool junction “shoe” 80 (can be a hot junction shoe if the current is reversed). The P and N legs are separately connected at a second end to connector wires 84, 85. The ends of the thermoelectric elements are referred as junctions. The other set of junctions are the reference junctions 89, 90. The manner in which P and N legs are formed and the Peltier effects is known to those skilled in the art and forms no part of the present invention.

Referring to FIG. 3, thermoelectric cooling of the contact junctions 86, 87 occurs when an electrical current is passed through the legs in the N to P direction, which is controlled by the power switch control 48 at the handle 46. The reference junctions 89, 90 experience heating when this electrical current is passed through the legs. Additional Joulean heating occurs in the legs 82, 83 because of the internal electrical resistance of the legs. This Joulean heating diminishes the cooling of the cold junction shoe 80. There is a need to disperse the heat generated at the legs and at the reference junctions 89, 90. The catheter of the present invention may comprise a re-circulating cryogenic fluid pathway adapted for absorbing and dissipating heat that is generated as a result of Peltier effect.

In one operation mode, an extendable guidewire 88 may be advanced through a catheter sheath into a patient's artery. The guidewire 88 is directed down the artery with the use of X-rays to monitor its progress. A catheter with a thermoelectric therapeutic element 81 is extended over the length of the guidewire 88 to the lesion site for cryo-therapy and/or heat-therapy.

As referred to FIG. 2 of the present invention, the tip junction 71 is configured to be positioned for contacting the vulnerable plaque intimately. More particularly, the tip junction 71 may be configured as a concave configuration adapted for encircling the vulnerable plaque for a purpose of intimate contact. Depending on the shape and structure of a vulnerable plaque, other configuration for the tip junction may also be applicable, such as convex, semi-cylindrical, circular, elliptic hyperboloid, ellipsoid, oblate spheroid, hyperbolic paraboloid, elliptic paraboloid, and irregular configuration. In an alternate embodiment, the method further comprises applying radiofrequency current to the tip junction adapted for heating said vulnerable plaque. The mode of heating and cooling can be alternate, sequential or other pre-programmed mode.

As shown in FIG. 2, the electromotive elements 72 and 73 are configured and arranged so that the reference junctions 74, 75 are adjacent a surface of the catheter shaft that is opposite from the tip junction 72. By such an arrangement, the heat generated at the reference junctions 74, 75 is constantly swept away by the flowing blood stream. Therefore, the tip junction 71 can maintain its coolness effective for prolonged operation for rendering the vulnerable plaque less vulnerable. The tip junction may preferably be configured as a concave configuration adapted for encircling the vulnerable plaque for a purpose of intimate contact. Furthermore, the concave junction may comprise a plurality of needles or probes configured for penetrating into an inner part of said vulnerable plaque for enhanced energy transfer.

In other embodiments as shown in FIG. 2, the tip junction 71, the and/or the reference junctions 74, 75 can be positioned at about the surface of the catheter shaft for effective cooling or heating therapy. In another embodiment, the tip junction, and/or the reference junctions may be embedded within the catheter shaft for stability. The embedding process may include molding or other known process.

The cryogenic catheter of the present invention may be provided with a cryogenic fluid path to cool a distal contact section of the catheter. Alternately, the distal contact section is adapted to serve as a therapy element of the present invention. The distal contact section is generally configured to be positioned for contacting the vulnerable plaque intimately. U.S. Pat. No. 6,235,019 discloses a cryosurgical catheter having at least one cryogenic fluid path for tissue treatment, the entire contents of which are incorporated herein by reference.

The cryogenic catheter of the invention can be a component in a cryogenic system that further includes a cryogenic fluid supply in communication with the cryogenic catheter, and a fluid controller interposed between the cryogenic catheter and the cryogenic fluid supply for regulating the flow of the cryogenic fluid into the cryogenic catheter. The cryogenic fluid can be a gas or a liquid.

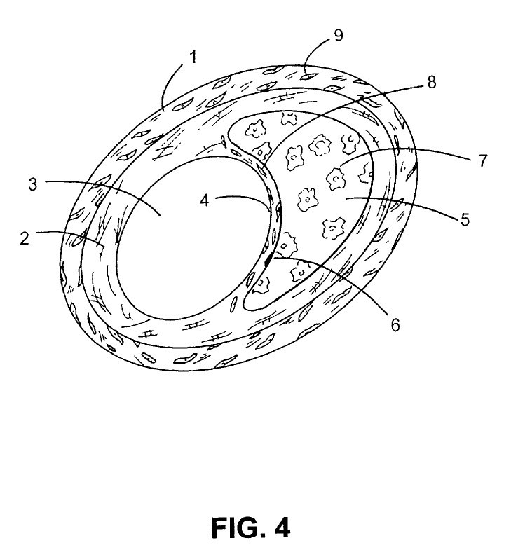

FIG. 4 shows a cross-sectional view of a vulnerable plaque for illustration purposes. In one embodiment, a vulnerable or risky plaque is generally believed to have a thin fibrous cap 4 on top of a lipid core 5. A blood vessel comprises an intima layer 2, a media layer 1 and an adventitia layer (not shown). In the process of plaque formation, a lipid core 5 may form inside the intima layer 2 and may either push the tissue layer outwardly or compress the lumen 3 of the blood vessel inwardly. Typically macrophage foam cells 7 around the lipid core or lipid pool 5 and the activated intimal smooth muscle cells 8 may tend to aggravate the plaque formation while T-lymphocytes 6 may also participate in the plaque formation process. The normal medial smooth muscle cells 9 maintain the media layer 1 at essentially its original shape or structure. The thin fibrous cap 4 may be prone to rupture due to shear stress, injury, hypertension, erosion, or other unknown reasons. During the early stage of plaque formation, inflammation is a major factor associated with vulnerable plaques, while at a later stage, atherosclerosis (or hardening) of the plaque dominates the pathological process.

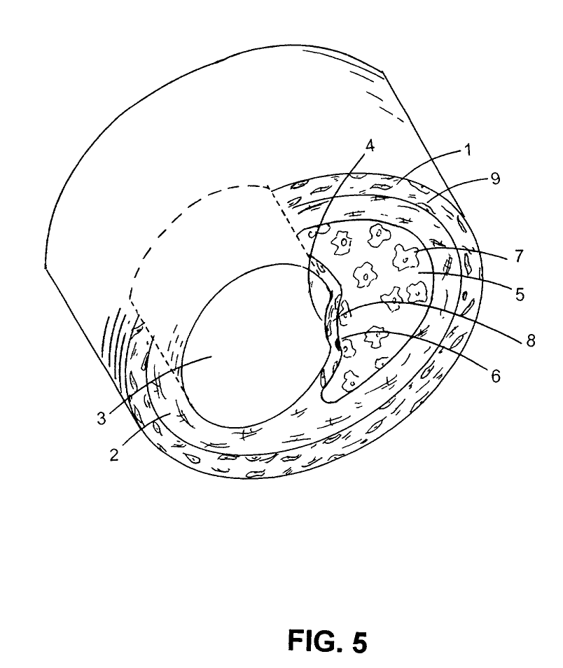

FIG. 5 shows a longitudinal cross-sectional view of a vulnerable plaque for illustration purposes. A vulnerable plaque might appear inside the blood vessel. The thin fibrous cap 4 at a vulnerable plaque site may become convex so that a therapeutic contact element with a concave surface to encircle the vulnerable plaque might effectively treat the tissue so as to reduce the chance of unexpected plaque rupture or fissure (the vulnerability).

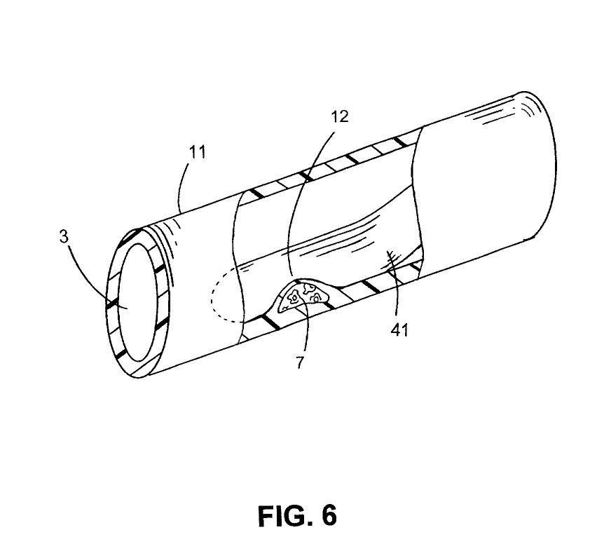

For further illustrations, FIG. 6 shows a perspective view of a blood vessel 11 having a vulnerable plaque 12. A medical catheter 41 of the present invention is inserted through an opening and the lumen 3 of the blood vessel 11 to the plaque site. The concave contact element is positioned and adapted for staying on top of the vulnerable plaque for applying cryo-therapy to render the vulnerable plaque less vulnerable.

Topol (J Invas Cardiol 12 (suppl B):2B-7B, 2000) studies the relationship of inflammation and embolization in ischemic heart disease. He reports that the inflamed artery culminates in a plaque fissure, erosion or rupture. The exposure of subendothelial matrix sets up platelet-thrombus deposition and the artery is markedly predisposed to micro-embolization and the potential for microcirculatory obstruction. He also reports that pathologic studies have unequivocally shown that vulnerable arterial plaque has less collagen, an increase in lipid pool constituency, and diminished fibrous cells, but a relatively high mononuclear cell count (monocytes and macrophages). Furthermore, the extracted tissue from vulnerable plaque is rich in expressing interleukin-6, tumor necrosis factor alpha and gamma interferon. These studies strongly confirm the presence of inflammation in affected coronary arteries with vulnerable plaque.

Formation of a lipid pool is suggested as a result of inflammatory process by some researchers. To mitigate the process or even to reverse the lipid-forming process, a cryo-therapy is proposed to substantially counter the inflammatory process. A cool therapy of lower than 36° C. is general applicable. More specifically, a temperature range of 36° C. to −30° C. is preferred, though other lower temperature may be equally applicable in the present invention. A temperature for cooling the vulnerable plaque in the range of about 36° C. to −30° C. may be sufficient to cool and solidify the lipid pool so as to render the vulnerable plaque less vulnerable. With regard to the general diffusion process, when the lipid concentration in the lipid pool is over-saturated under a cold environment, a reversal diffusion process might theoretically occur.

The fundamental lesion of atherosclerosis is the atheromatous or fibrofatty plaque, which may cause narrowing of the artery, predispose to thrombosis, calcify, lead to weakening of the muscle, and cause aneurysmal dilation. Atherosclerotic plaques and some vulnerable plaques as a sub-group are approximately rounded, raised lesions, usually off-white to white in color superficially, and perhaps a centimeter or less in diameter. The center of larger plaques may exude a yellow, grumous fluid.

Acute myocardial infarction is believed to be caused by rupture of an unstable coronary-artery plaque that appears as a single lesion on angiography (N Eng J Med 2000;343:915-922). However, plaque instability might be caused by pathophysiologic processes, such as inflammation, that exert adverse effects throughout the coronary vasculature and that therefore result in multiple unstable lesions. Goldstein et al. analyzed angiograms from patients for complex coronary plaques characterized by thrombus, ulceration, plaque irregularity and impaired flow to document the presence of multiple unstable plaques in patients with acute myocardial infarction. Single complex coronary plaques were identified in about 60% patients and multiple complex plaques in about 40% patients. They concluded that patients with acute myocardial infarction might harbor multiple complex coronary plaques that are associated with adverse clinical outcomes. With the present invention, a medical catheter can be employed to treat a plurality of vulnerable plaques.

The method for rendering a vulnerable plaque of a patient less vulnerable comprising cooling said vulnerable plaque, wherein the vulnerable plaque is characterized by a lipid pool under a thin cap of the vulnerable plaque. The method may further comprise detecting said vulnerable plaque prior to treatment, wherein the detecting method may comprise monitoring the temperature of the tip junction in accordance with a thermocouple principle. The method may further comprise a step for monitoring the temperature of the tip junction in accordance with a thermocouple principle, optionally using the tip junction of the medical catheter of the present invention.

From the foregoing description, it should now be appreciated that a medical device comprising incorporating cryogenic therapy sufficiently to render a focal vulnerable plaque less vulnerable has been disclosed. While the invention has been described with reference to a specific embodiment, the description is illustrative of the invention and is not to be construed as limiting the invention. Various modifications and applications may occur to those who are skilled in the art, without departing from the true spirit and scope of the provisional application.

What is claimed is:

1. A method for rendering a vulnerable plaque of a patient less vulnerable comprising cooling said vulnerable plaque.

2. The method according to claim 1, wherein a temperature for cooling said vulnerable plaque is no higher than 36° C. that is effective to render the vulnerable plaque less vulnerable.

3. The method according to claim 1, the method further comprising detecting said vulnerable plaque prior to cooling treatment.

4. The method according to claim 3, wherein a technique for detecting said vulnerable plaque is for monitoring temperature of said vulnerable plaque.

5. The method according to claim 1, wherein a temperature for cooling said vulnerable plaque is in the range of about 36° C. to −30° C. that is sufficient to cool and solidify the lipid pool.

6. The method according to claim 1, wherein the vulnerable plaque is characterized by a lipid pool under a thin cap of the vulnerable plaque.

7. The method according to claim 5, the method further comprising detecting said vulnerable plaque prior to treatment.

8. The method according to claim 5, wherein said cooling is accomplished with a cryogenic catheter, and wherein said cryogenic catheter is provided with a cryogenic fluid path to cool a distal contact section of the catheter, said distal contact section being configured to be positioned for contacting the vulnerable plaque intimately.

9. The method according to claim 8, wherein the distal contact section is configured as a concave configuration adapted for encircling the vulnerable plaque for a purpose of intimate contact.

10. The method according to claim 1, wherein said cooling is accomplished with a cryogenic catheter, said cryogenic catheter comprising elements of different electromotive potential conductively connected at a tip junction, the method further comprising passing an electrical current through said elements to reduce a temperature of said tip junction in accordance with the Peltier effect adapted for thermoelectric cooling said vulnerable plaque.

11. The method according to claim 10, wherein the tip junction is configured to be positioned for contacting the vulnerable plaque intimately.

12. The method according to claim 10, wherein the tip junction is configured as a concave configuration adapted for encircling the vulnerable plaque for a purpose of intimate contact.

13. The method according to claim 10, the method further comprising monitoring the temperature of the tip junction in accordance with a thermocouple principle.

14. The method according to claim 10, the method further comprising applying radiofrequency current to the tip junction adapted for heating said vulnerable plaque.

15. A medical catheter for rendering a vulnerable plaque of a patient less vulnerable, said medical catheter comprising:
elements of different electromotive potential conductively connected at a thermoelectric tip junction disposed at a distal section of the medical catheter, wherein the thermoelectric tip junction is adapted for contacting said vulnerable plaque;
a first electrical wire and a second electrical wire supplying an electrical current through said elements to reduce temperature of said tip junction in accordance with the Peltier effect adapted for thermoelectric cooling said vulnerable plaque.

16. The medical catheter according to claim 15, wherein the tip junction is configured as a concave configuration adapted for encircling the vulnerable plaque for a purpose of intimate contact.

17. The medical catheter according to claim 16, wherein said concave junction comprises a plurality of needles or probes configured for penetrating into an inner part of said vulnerable plaque.

18. The medical catheter according to claim 15, wherein the tip junction is configured and adapted for encircling the vulnerable plaque for a purpose of intimate contact, configuration of said tip junction being selected from a group consisting of semi-cylindrical, circular, elliptic hyperboloid, ellipsoid, oblate spheroid, hyperbolic paraboloid, elliptic paraboloid, and irregular configuration.

19. The medical catheter according to claim 15, the catheter further comprising a re-circulating cryogenic fluid pathway adapted for absorbing heat that is generated as a result of Peltier effect.

20. A method for rendering a vulnerable plaque of a patient less vulnerable, the method comprising cooling said vulnerable plaque using a cryogenic catheter, wherein the cryogenic catheter comprises elements of different electromotive potential conductively connected at a thermoelectric tip junction disposed at a distal section of the medical catheter, wherein the thermoelectric tip junction is adapted for contacting said vulnerable plaque, a first electrical wire and a second electrical wire supplying an electrical current through said elements to reduce temperature of said tip junction in accordance with the Peltier effect adapted for thermoelectric cooling said vulnerable plaque, and wherein configuration of said tip junction is selected from a group consisting of semi-cylindrical, circular, elliptic hyperboloid, ellipsoid, oblate spheroid, hyperbolic paraboloid, elliptic paraboloid, and irregular configuration.
